(19) United States
(12) Patent Application Publication
(10) Pub. No.: US 2010/0191073 A1
(43) Pub. Date: Jul. 29, 2010

(54) SYSTEM FOR MONITORING CHEMOTHERAPY-ASSOCIATED ADVERSE DRUG REACTIONS

(75) Inventors: Lionel Tarassenko, Oxford (GB); Oliver John Gibson, Abingdon (GB); M. E. Larsen, Oxford (GB)

Correspondence Address: NIXON & VANDERHYE, PC, 901 NORTH GLEBE ROAD, 11TH FLOOR, ARLINGTON, VA, 22203, US

(73) Assignee: T + MEDICAL LIMITED, Abingdon (GB)

(21) Appl. No.: 12/451,393

(22) PCT Filed: May 14, 2008
(86) PCT No.: PCT/GB2008/001671

§ 371 Date: Apr. 2, 2010

(30) Foreign Application Priority Data
May 14, 2007 (GB) 0709248.9

(51) Int. Cl. A61B 5/00; G06Q 10/00; G06Q 50/00

(52) U.S. Cl. 600/301; 705/2

(57) ABSTRACT

A system adapted to monitor adverse drug reactions in cancer patients undergoing chemotherapy which comprises a patient-based data terminal 3 such as a mobile telephone adapted to provide for periodic entry by the patient of data on a plurality of predetermined adverse reactions. The data is processed for display to the patient and for transmission to a remove server 7. The data is also processed to generate red or amber alerts which may be displayed to the patient and sent by the server 7 to a pager 13 held by the healthcare professional. The server 7 processes the received data for display and review via a secure web page. Alerts may be of different levels of urgency with urgent alerts being sent immediately to the pager 13, but less urgent alerts being sent in batches at regular intervals.

The present invention relates to a system for monitoring chemotherapy-associated adverse drug reactions (side-effects), in particular for cancer patients who are undergoing chemotherapy as outpatients, i.e. based largely in their own home.

Cancer patients undergoing treatment by use of cytotoxic drugs (referred to here as chemotherapy) are now commonly based at home during the period of treatment. A typical chemotherapy regime is based on a number, typically 8-12, of three week treatment cycles. Each cycle consists of the administration of the cytotoxic drug, commonly at an outpatient clinic, following which the patient returns home. Over the next 10-14 days, as the drug takes effect, the patient typically suffers a variety of adverse drug reactions (ADRs—commonly known as side-effects) of varying seriousness. After two weeks the patient will tend to start feeling better and has a week of respite before the next administration of the drug. Common adverse reactions to chemotherapy include febrile neutropenia, diarrhoea, nausea, vomiting and mucositis. Patients may suffer very severely from these and some chemotherapy regimens have toxic death rates of the order of 0.8 to 2.2%.

Good management of the side effects of chemotherapy can reduce their severity and thus make the process less unpleasant for the patient. Currently management of side effects is handled by a variety of measures including patient education with pre-chemotherapy discussion, information leaflets, patient-held diaries and support from healthcare professionals. However, unless the patient contacts the healthcare professional directly, or has an intervening appointment set, their side effects will only be discussed at the next clinic appointment. Commonly, therefore, side effects for one cycle are not discussed with a healthcare professional until just before the start of the next cycle. Closer involvement of healthcare professionals, while desirable, is expensive, and it is now being recognised that empowering patients to manage their own health has a positive effect on the outcomes of therapy and improving the psychological state of the patient.

According to the present invention there is provided a system adapted to monitor side-effects in cancer patients undergoing chemotherapy, the system comprising:

a patient-based data terminal adapted to provide for periodic entry by the patient of data on a plurality of predetermined adverse drug reactions, to process the entered data, to display the processed data to the patient and to transmit the entered data;

a server adapted to receive the data transmitted from the data terminal, to process the data, and to transmit the processed data for display;

a remote terminal adapted to receive the processed data from the server and display it; and

an alerting device (e.g. a pager) for use by a healthcare professional;

wherein the patient-based data terminal and the server both process the data independently based on the same criteria to generate selectively from the entered data an alert of a first or second type, the patient-based data terminal displays said alert to the patient, the server collects alerts of the first type into batches and regularly transmits the batches to said alerting device, and the server immediately transmits alerts of the second type individually to said alerting device.

The invention therefore provides for the patient to monitor their condition themselves according to a pre-defined set of expected adverse reactions, and to enter that data onto their own data terminal. The data is processed to provide feedback to the patient in the form of a display of their current and past condition and, optionally, advice on action to be taken. The data is also transmitted, preferably via the internet, to a secure server which stores the data and independently processes it using the same criteria as the patient-based data terminal. The server is adapted to generate alerts of a first type (amber) or a second type (red) on the basis of predefined criteria. Amber alerts are collected into batches and transmitted periodically, say three times a day, to the pager of a healthcare professional. Red alerts are sent immediately to the pager of the healthcare professional. The server also makes the data available for viewing by the healthcare professional on a secure web page.

Thus in response to the red alert the healthcare professional can immediately access the web page to check the patient's data and can contact the patient. On receipt of the batches of amber alerts the healthcare professional can, at an appropriate time, check these and decide on appropriate action.

The processing of the data at the patient-based data terminal using the same criteria as at the server allows the patient to see when a red alert has been generated. The terminal can display information advising the patient to take further action, for example to take or stop taking medication or, in the case of a red alert, to contact the healthcare professional if the patient is not themselves contacted within a pre-set time of the alert being generated. In the case of a patient's condition not generating any alert, the patient can still feel reassured that their condition is being monitored and is satisfactory. On the other hand, the healthcare professionals can target their attention to those patients needing it.

The patient-based data terminal may conveniently be a mobile telephone or similar telephony-enabled personal digital assistant. This means that the patient is using technology which will be very familiar to them and which is robust and user-friendly.

The data entered by the patient preferably consists of a combination of measured values, such as temperature, white blood cell count, blood pressure, heart rate, etc., together with subjective self-assessments as to the severity of predetermined adverse reactions such as feelings of nausea, episodes of diarrhoea, vomiting, mucositis, etc. These self-assessments may be entered by the patient in response to the display of a series of questions.

The alerts are preferably generated based on data entered over a plurality of successive data entry periods. For example, patients may be required to enter data on their condition twice daily and thus each data entry period consists of 12 hours. Each adverse reaction may be monitored over a suitable number of successive periods and the criteria for generation of an alert, and the type of alert, can be based on the severity of the adverse reaction encountered over set numbers of those periods.

Alerts may also be generated in the case of lack of data entry, and in particular by certain combinations of adverse reactions occurring followed by a lack of data entry.

As compared with current practice in chemotherapy, the system of the present invention provides for real time monitoring and response for patients undergoing chemotherapy at home.

It will be appreciated that the invention may be embodied in computer programs (software applications) stored and executed on the patient-based data terminal and the server, and thus that the invention extends to such computer programs.

The invention will be further described by way of example with reference to the accompanying drawings in which:

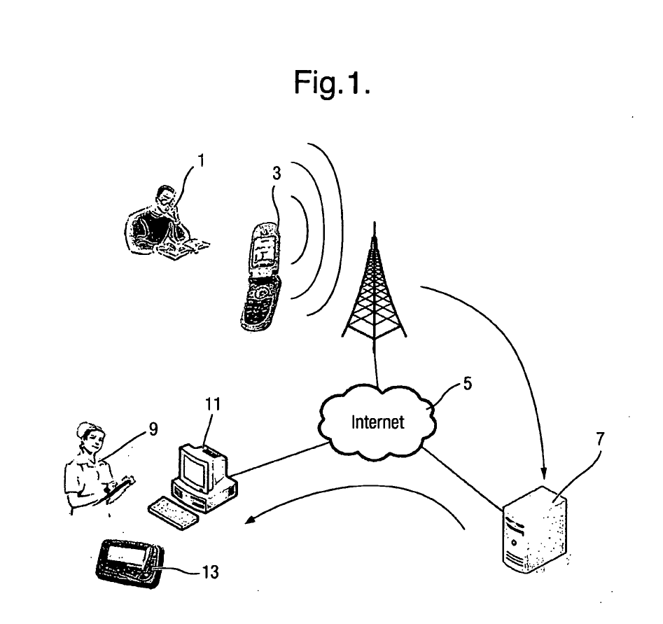

FIG. 1 schematically illustrates an embodiment of the present invention;

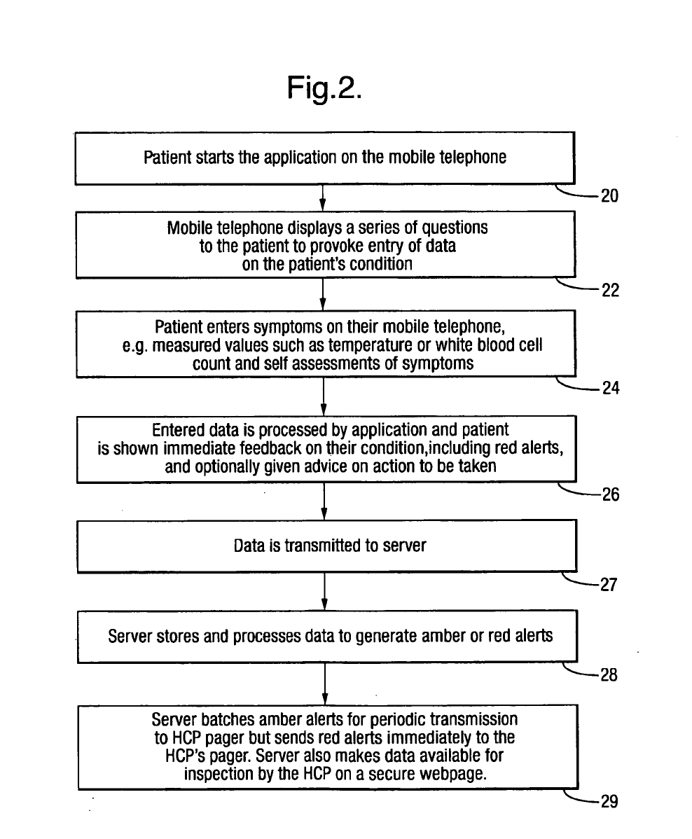

FIG. 2 is a flow diagram of the overall process of an embodiment of the present invention;

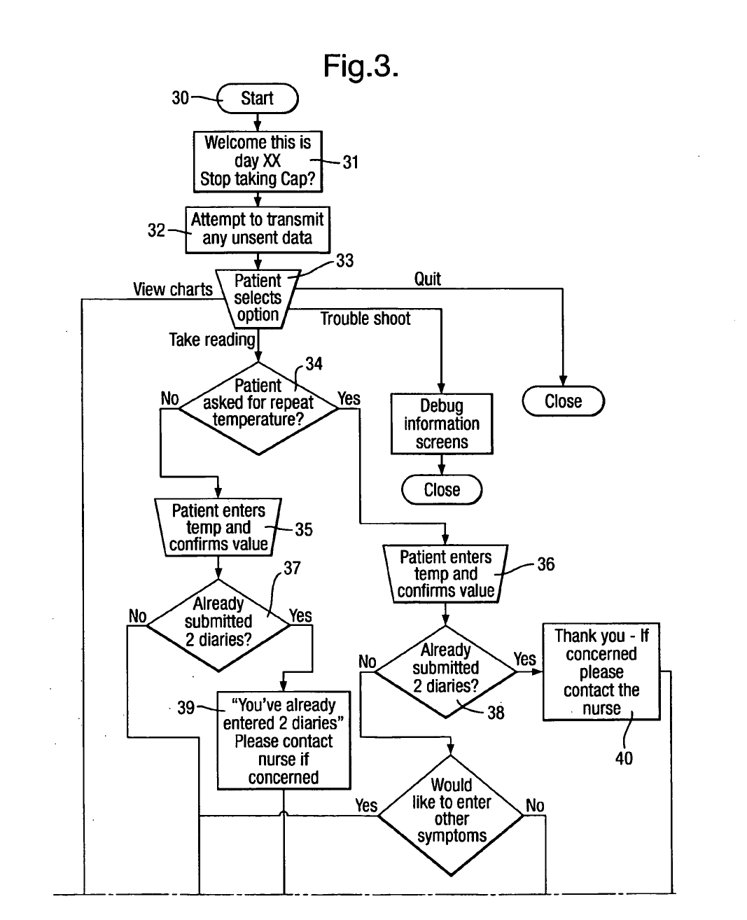

FIG. 3 is a flow diagram of the software on a patient-based data terminal of one embodiment of the invention;

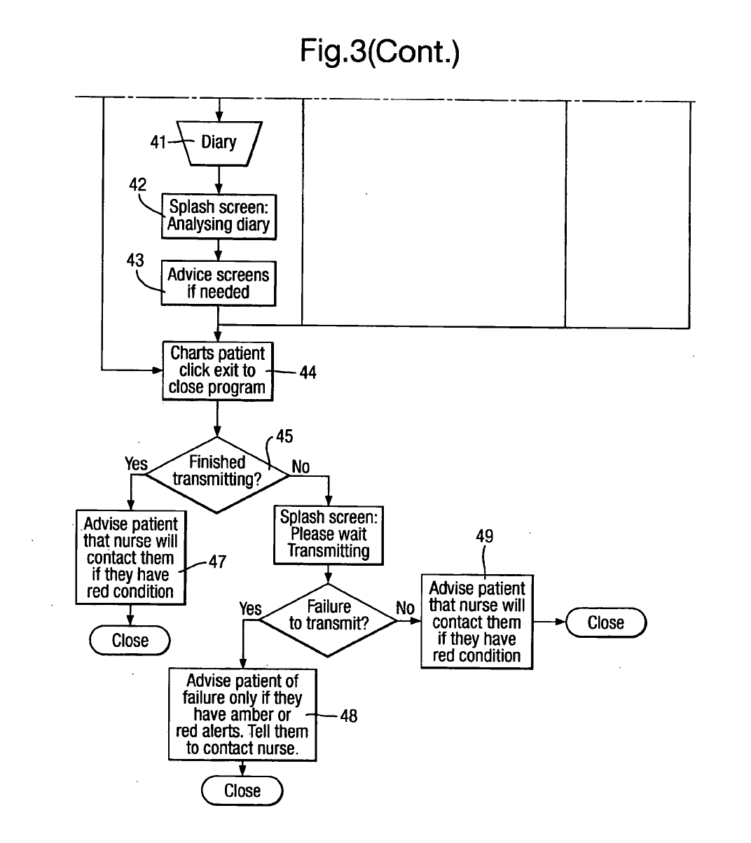

FIG. 4 is a flow diagram illustrating the response of the patient-based data terminal to an input temperature measurement;

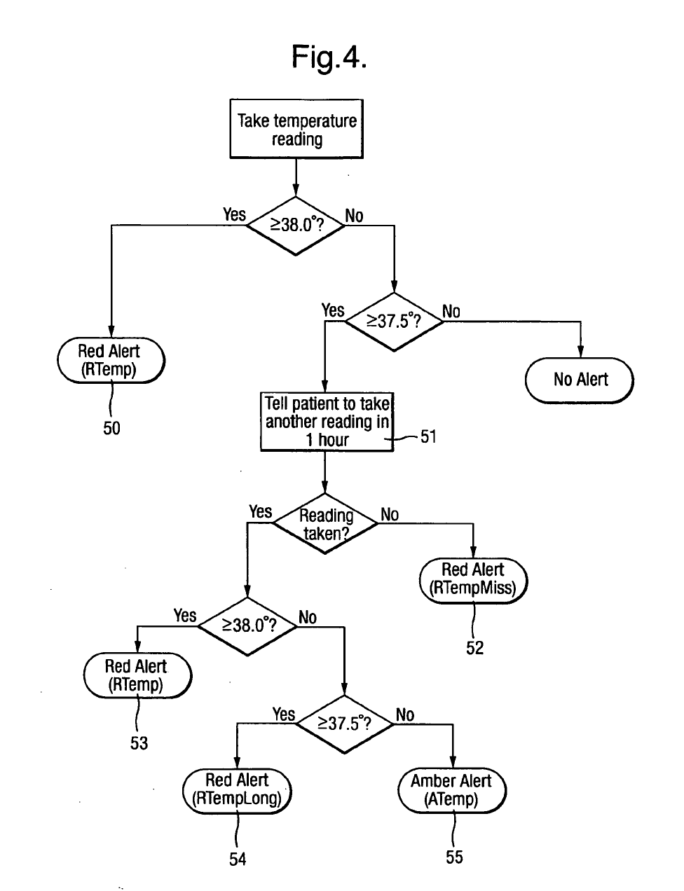

FIG. 5 illustrates examples of the questions displayed to a patient on the patient-based data terminal to provoke entry of the symptoms;

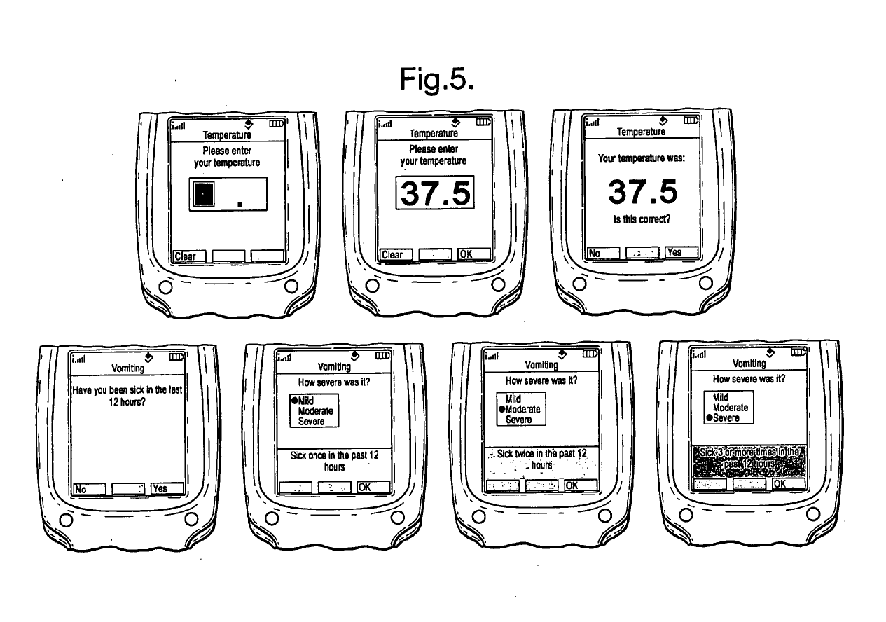

FIG. 6 illustrates a display to the patient on the patient-based data terminal of their symptoms;

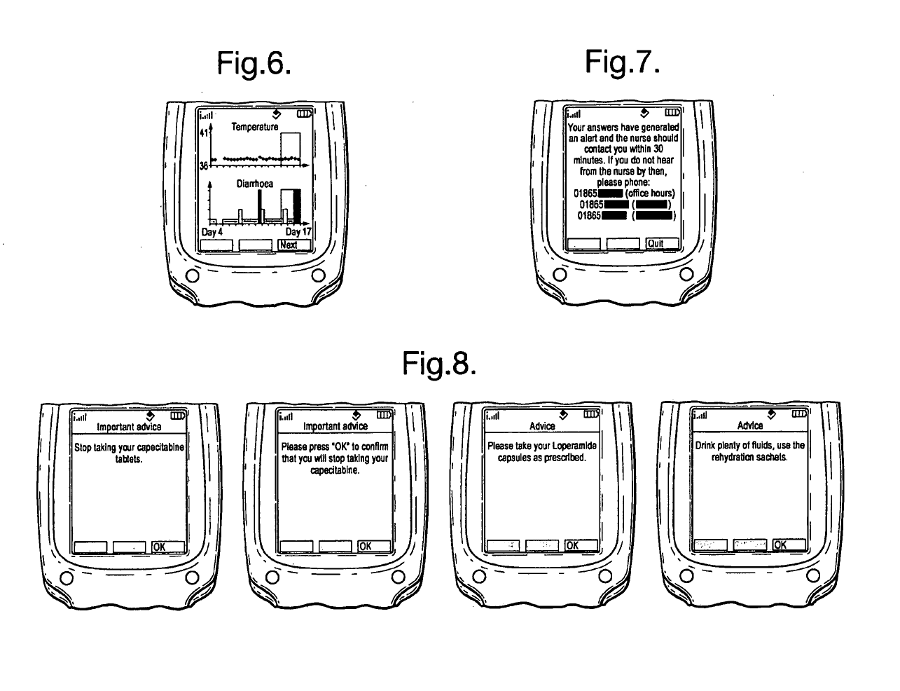

FIG. 7 illustrates the display of an alert to a patient in one embodiment of the invention;

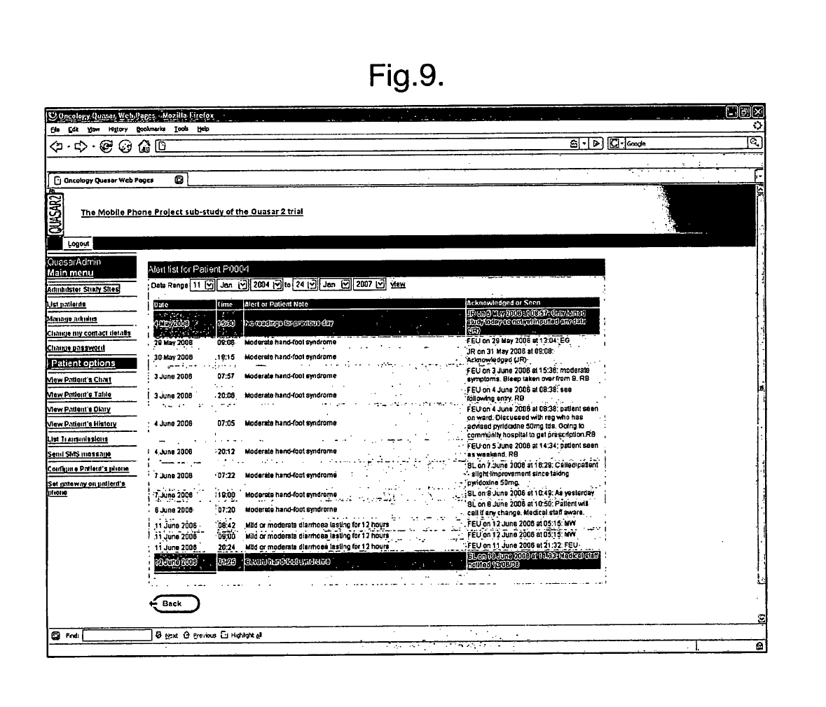

FIG. 8 illustrates the display of advice to a patient following entry of data; and

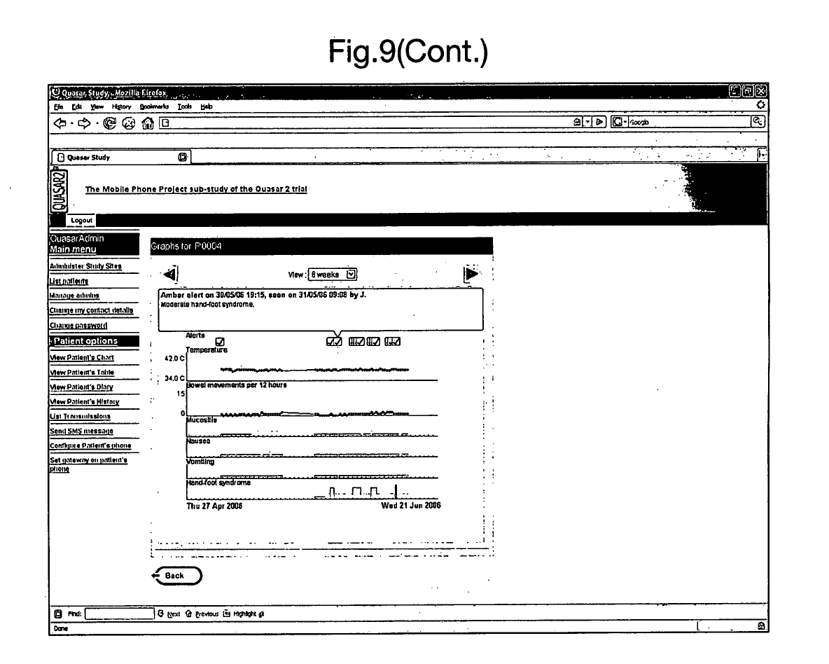

FIG. 9 illustrates the web page display of the patient-entered data in one embodiment of the invention.

FIG. 1 is a schematic diagram of a system of one embodiment of the present invention. Patients 1 are each provided with a data terminal 3 in this embodiment illustrated as a mobile telephone 3, which is preloaded with a software application for allowing data entry, for processing the data and for displaying it to the patient and transmitting it over the internet 5 to a server 7. The server 7 is provided with software which stores the data, processes it in the same way as the software on the mobile telephone 3 to generate alerts as will be discussed below, and also processes it for display as a web page viewable by a healthcare professional 9 using a remote terminal 11. The server 7 transmits alerts based on the data entered by the patient to a pager 13 held by the healthcare professional 9 in order to alert the healthcare professional 9 to review the patient's condition and take appropriate action such as contacting the patient.

FIG. 2 illustrates the overall process flow. In step 20 the patient starts the application on the mobile telephone 3. In the case of the telephone being dedicated to this use, the software application may be set up to start automatically on powering up of the mobile telephone. When the application has started it displays at step 22 a series of questions to the patient 1 to cause the patient 1 to enter at step 24 data on a variety of predetermined symptoms expected or possible with their chemotherapy. For example in an international trial evaluating chemotherapy for patients with colon cancer, patients were asked to measure and enter their temperature, and also symptoms related to nausea, vomiting, mucositis, diarrhoea/bowel movements and hand/foot syndrome. FIG. 5 illustrates examples of such questions. Preferably the questions are simplified for display on the mobile telephone screen. Table 1 below illustrates examples of the flow of displayed questions for the patient's self-assessment in the trial mentioned above.

TABLE 1

| No. | Description |
|---|---|
| V30.1 | Diarrhoea |
| | B1. How many times have you opened your bowels in the last |
| | 12 hours? [go to C1] |
| | Mucositis |
| | C1. Have you noticed any problems with your mouth or |
| | throat in the last 12 hours? |
| | Yes [go to C2] |
| | No [go to D1] |
| | C2. How severe were they? |
| | Mild - redness, tingling, can eat and drink as usual |
| | Moderate - mouth ulcers, or can eat only a soft diet and drink |
| | Severe - mouth ulcers and\or bleeding, or unable to eat |
| | and drink sufficiently |
| | [go to D1] |
| | Nausea |
| | D1. Have you felt sick in the last 12 hours? |
| | Yes [go to D2] |
| | No [go to E1] |
| | D2. How severe was it? |
| | Mild - loss of appetite, but eating and drinking normally |
| | Moderate - eating and drinking a little less than normal |
| | Severe - only eating and drinking small amounts or not able |
| | to eat and drink at all |
| | [go to E1] |
| | Vomiting |
| | E1. Have you been sick in the last 12 hours? |
| | Yes [go to E2] |
| | No [go to F1] |
| | E2. How severe was it? |
| | Mild - sick once in the past 12 hours |
| | Moderate - sick twice in the past 12 hours |
| | Severe - sick 3 or more times in the past 12 hours. |
| | [go to F1] |
| | Bleeding |
| | F1. Have you experienced any bleeding in the last 12 hours? |
| | Yes [go to F2] |
| | No [go to G1 if evening timeslot, else end] |
| | F2. Has the bleeding lasted for more than 30 minutes in total? |
| | Yes [go to G1 if evening timeslot, else end] |
| | No [go to G1 if evening timeslot, else end] |
| | Fatigue (only asked in the evening timeslot) |
| | G1. Have you felt fatigued/tired in the last 24 hours? |
| | Yes [go to G2] |
| | No [end] |
| | G2. How severe was the fatigue? |
| | Mild - up and about, and still able to carry out light work |
| | Moderate - unable to work, but generally up and about |
| | Severe - confined to bed or chair for most of waking hours. |
| | [end] |

As illustrated in FIG. 5, for their responses, patients are able to select as the severity of their reaction grade 1 (“mild”) or 2 (“moderate”), with grades 3 and 4 toxicities combined into a “severe” category. The subjective assessments illustrated in FIG. 5 are set such that the default selection is the worst possible case. This means that the system fails safe in the event of misuse of the diary. The patient chooses between the displayed options, e.g. the categories of “mild”, “moderate” and “severe”, by use of the mobile telephone cursor keys and, as illustrated in FIG. 5, the display also displays to the patient the definition of each of the categories so that the patient can judge their condition correctly. This improves the reliability of the data.

The application is also designed to allow entry of numerical data using the mobile telephone keypad and preferably checks that the entered values are realistic. In this embodiment the application requires the patient to confirm the entered value as correct before moving onto the next question.

After entry of all the data the software application processes the data at step 26 to display the patient's cumulative toxicity charts as illustrated in FIG. 6. While these charts are displayed the application automatically transmits, at step 27, the entered data via a secure GPRS connection to the dedicated server 7. The data sent to the server 7 consists of the: time, temperature, symptom scores, patient identity number, and data flow verification codes including, for example, the treatment cycle number to check synchronization with the server 7. In the event of a failure to transmit (after a certain time-out period) the data is stored for retransmission next time the software application is started.

The software application on the mobile telephone also checks the data to generate amber or red alerts according to stored criteria. Table 2 below illustrates the toxicity alert criteria for the colon cancer chemotherapy trial mentioned above (the same criteria are used at the server 7).

TABLE 2
Toxicity alert criteria

| Alert | Condition | Parameter |
|---|---|---|
| Amber | Borderline pyrexia with normal second reading | reading in the range 37.5-37.9° C. and 2^{nd} |
| | | reading (after 1 hour) is <37.5° C. |
| | Mild or moderate diarrhoea lasting for 12 hrs | total number of bowel movements over |
| | | baseline in last two readings ≧4 |
| | Severe mucositis lasting for 24 hrs | severe mucositis in 2 or more of 3 readings |
| | Moderate mucositis lasting for 48 hr | moderate or severe mucositis in 3 or more |
| | | of 5 readings |
| | Severe nausea lasting for 48 hrs | severe nausea in 3 or more of 5 readings |
| | Moderate or severe vomiting lasting for 24 hrs | moderate or severe vomiting in 2 or more of |
| | | 3 readings |
| | Moderate Hand-Foot Syndrome | moderate hand foot syndrome in current |
| | | reading |
| Red | No readings for previous day | no readings received in the last 30 hours |
| | Many sufficiently concerning amber alerts in | any combination of ambers 2, 5 or 6, in 4 or |
| | previous 48 hours | more of 5 readings |
| | Pyrexia | current temperature reading is 38.0° C. or |
| | | above |
| | Borderline pyrexia for 12 hours | borderline pyrexia where 2^{nd} reading is also |
| | | 37.5-37.9° C. |
| | Borderline pyrexia and second reading not | borderline pyrexia where 2^{nd} reading is not |
| | known | taken within 90 minutes |
| | Mild or moderate diarrhoea lasting for 36 hrs | total number of bowel movements over |
| | | baseline in last 4 readings ≧8 |
| | Severe diarrhoea | total number of bowel movements over |
| | | baseline in current reading ≧4 |
| | Moderate diarrhoea and severe nausea | number of bowel movements over baseline ≧2 |
| | | and severe nausea |
| | Moderate diarrhoea and moderate or severe | number of bowel movements over baseline ≧2 |
| | vomiting | and moderate or severe vomiting |
| | Moderate diarrhoea and severe mucositis | number of bowel movements over baseline ≧2 |
| | | and severe mucositis |
| | Severe mucositis and moderate or severe | severe mucositis and moderate or severe |
| | vomiting | vomiting in the current reading |
| | Severe mucositis and severe nausea | severe mucositis and severe nausea in the |
| | | current reading |
| | Severe vomiting for 12 hours | severe vomiting in 2 readings |
| | Severe hand-foot syndrome | severe hand-foot syndrome in the current |
| | | readings |
| | Moderate hand-foot syndrome lasting for 24 | moderate (not severe) hand-foot syndrome |
| | hours | in all last 3 readings |

The application on the telephone 3 is also adapted to display advice to the patient, based on the entered data, such as to adjust their medication or take additional medication or to stop taking medication. Examples are illustrated in FIG. 8.

Further, if an amber alert is generated for four out of five consecutive 12 hour time slots, it is automatically escalated to a red alert. In the case of a red alert the software application displays to the patient an indication that a red alert has been generated as illustrated in FIG. 7 and indicates that they will be contacted by a healthcare professional within a certain time, or, if not, that they should contact their healthcare professional. Amber alerts are not displayed to avoid worrying the patient.

As illustrated at step 28 of FIG. 2, the server 7 also runs a software application to process the data in the same way as the application on the mobile telephone i.e. using the criteria of Table 2. In order to allow for failure of data entry by the patient, the server software application generates a red alert if data is not entered for more than 24 hours. The server software is adapted to send an SMS message to the patient prompting them to submit data in addition to the generation of the red alert.

At step 29, amber alerts are sent by the server 7 in batches to the healthcare professional's pager at regular times during the day, for example, 9 a.m., 1 p.m. and 3.30 p.m., seven days a week. Amber alerts indicate that the patient is experiencing some difficulties but that these are not severe or life threatening. Red alerts, however, indicate that the patient is pyrexial and/or experiencing symptoms that are severe or life threatening. Red alerts are therefore sent by the server 7 to the healthcare professional's pager 13 immediately on receipt and processing of the data. The red alert indicates the identity of the patient. The server 7 additionally processes the entered data into a form suitable for viewing on a secure web page as illustrated in FIG. 9. On receiving a red alert, or at an appropriate time when handling amber alerts, the healthcare professional 9 can access the web pages using a remote terminal 11 and take appropriate action such as contacting the patient 1.

FIG. 3 illustrates the flow of the software on the patient-based data terminal 3 in a particular embodiment of the invention. After starting 30, the application displays the current day of the treatment cycle and optionally advice as to whether medication is to be taken or not. The display of the day of the cycle is based on the counter reset by the healthcare professional at each clinic appointment. In step 32 the application sends any previously entered unsent data (for example if a connection was not previously available). In step 33 the patient may select between the options of viewing charts of their condition, taking readings (i.e. entering data), troubleshooting or quitting. In the case of entering data, in step 34, following entry of the patient's temperature, it is decided whether the patient should be asked to check their temperature again within a short time, for example one hour, or not. If not, the patient is asked to confirm the entered temperature at step 35, but if so a message is displayed asking the patient to repeat the temperature reading again, for example within an hour, and the repeat temperature is entered at step 36.

In steps 37 to 40 it is checked whether the patient has already entered two sets of data for that day. The system is designed to prevent patients entering more than two sets of data and in the event that they try to do so the patient is asked to contact their healthcare professional. Steps 41 to 43 allow the patient to enter data on the other symptoms they are experiencing and in step 44, following completion of data entry, the charts showing the patient's condition are displayed. During this time the data terminal 3 transmits the data, if a connection is available, to the remote server 7. On checking at step 45 that the transmission is finished the patient can be advised at steps 47 and 49 that they will be contacted if they have a red alert, or can be advised at step 48 of the failure to transmit and the need to contact a healthcare professional if they have a red alert.

FIG. 4 illustrates the processing of the temperature reading. As will be understood from FIG. 4 if the temperature is greater than or equal to 38° C. a red alert is generated in step 50. If the temperature is borderline, i.e. between 37.5° C. and 38° C., the patient is requested in step 51 to take another reading within one hour. If this reading is not taken then a red alert is generated at step 52, or if the temperature has risen above 38° C. a red alert is generated at step 53. Otherwise if the temperature is still above 37.5° a red alert is generated at step 54, but if not an amber alert is generated at step 55.

The cumulative toxicity displays, one of which is illustrated in FIG. 6 for example, show the last 21 days' worth of data. Preferably data from the current medication cycle is shown with a different coloured background from older data. Each day is separated into two time slots to allow display of both 12 hour data entry periods.

As mentioned above alerts may be generated as a result of checks on the data, or lack of data. Checks for lack of data (compliance checks) are preferably made by the server software twice a day, and the current and two former timeslots are analysed. If no data is found then a red alert is generated. Time dependent alerts (24 or 48 hours of symptoms) are also generated if there has been no data recorded within the current or previous time, but previous timeslots contain the requisite symptoms (i.e. a lack of data is regarded as a worst case).

It will therefore be appreciated that red alerts generated as a result of data entered by the patient are transmitted immediately on receipt and processing of that data at the server 7 to the healthcare professional's pager 13. On the other hand red alerts generated by lack of data are transmitted when they are generated at the regular compliance checks on the server 7.

1.-12. (canceled)

13. A system adapted to monitor side-effects in cancer patients undergoing chemotherapy, the system comprising:
a patient-based data terminal adapted to provide for periodic entry by the patient of data on a plurality of adverse drug reactions, to process the entered data, to display the processed data to the patient and to transmit the entered data;
a server adapted to receive the data transmitted from the data terminal, to process the data, and to transmit the processed data for display;
a remote terminal adapted to receive the processed data from the server and display it; and
an alerting device for use by a healthcare professional;
wherein the patient-based data terminal and the server both process the data independently based on the same criteria to generate selectively from the entered data an alert of a first or second type, the patient-based data terminal displays said alert of the second type to the patient, the server collects alerts of the first type into batches and regularly transmits the batches to said alerting device, and the server immediately transmits alerts of the second type individually to said alerting device.

14. A system according to claim 13 wherein the alerting device is a pager.

15. A system according to claim 13 wherein the patient-based data terminal is a mobile telephone or telephony-enabled personal digital assistant.

16. A system according to claim 13 wherein the server makes processed data available as a secure web page accessible by the healthcare professional by use of the remote terminal.

17. A system according to claim 13 wherein the server is adapted to send said batches to said alerting device a plurality of times a day.

18. A system according to claim 13 wherein said data on a plurality of predetermined adverse drug reactions, comprises measured values and subjective self-assessments by the patient.

19. A system according to claim 18 wherein said patient-based data terminal is adapted to display a series of questions to the patient to provoke entry of said measured values and subjective self-assessments.

20. A system according to claim 18 wherein said measured values are selected from temperature, white blood cell count and blood pressure.

21. A system according to claim 13 wherein said alerts are based on data entered in a plurality of successive data entry periods.

22. A system according to claim 13 wherein said alerts are based on lack of data entry in a plurality of successive data entry periods.

23. A system according to claim 13 wherein said alerts are based on predetermined criteria for data entered in a plurality of successive data entry periods followed by lack of data entry.

24. A computer-readable storage medium having tangibly encoded thereon a computer program executable on a patient-based data terminal or on a server for use in the system of claim 13.
